(19) United States
(12) Patent Application Publication
(10) Pub. No.: US 2024/0245868 A1
(43) Pub. Date: Jul. 25, 2024

(54) AUTOINJECTOR HAVING A ROTATING DISPLAY

(71) Applicant: Ypsomed AG, Burgdorf (CH)

(72) Inventors: Gabriel Kalbermatter, Burgdorf (CH); Jürg Hirschel, Bern (CH); Nicolas Gruenig, Courtepin (CH)

(21) Appl. No.: 18/623,607

(22) Filed: Apr. 1, 2024

Related U.S. Application Data
(63) Continuation of application No. PCT/EP2022/076929, filed on Sep. 28, 2022.

(30) Foreign Application Priority Data
Oct. 7, 2021 (EP) 21201392.4

(51) Int. Cl. A61M 5/32

(52) U.S. Cl. CPC A61M 5/3257; A61M 5/3243

(57) ABSTRACT

An autoinjector having a housing, a product container axially fixed in the housing, a torsion spring, a drive element, a propulsion member, and a needle protection sleeve which, when the autoinjector is pressed against a point of injection, carries out an actuation movement in the proximal direction. The autoinjector includes a display for signaling the progression or persistence of a discharge, with a display element, which is driven by the drive element, rotates about the longitudinal axis and has an optical contrast pattern, and a transparent window at the proximal end of the autoinjector, through which the rotating contrast pattern is visible. The window is completely circumferential by 360° about the longitudinal axis, i.e., is not interrupted by a frame or web parallel to the longitudinal axis, which otherwise creates a dead angle for the view on the display element.

### CROSS-REFERENCE TO RELATED APPLICATIONS

This application is a continuation of International Patent Application No. PCT/EP2022/076929, filed Sep. 28, 2022, which claims priority to European Patent application Ser. No. 21201392.4, filed Oct. 7, 2021, each of which is incorporated by reference herein, in the entirety and for all purposes.

### TECHNICAL FIELD

Medical injection devices for administering liquid substances—in particular, medicaments or medical substances, such as insulin and hormone preparations are configured as single use autoinjectors with an energy store for ejecting a predetermined dose from a product container.

### BACKGROUND

Injection devices or injection apparatuses for the simplified administration of a substance comprise, inter alia, so-called autoinjectors which have an energy store with which the discharge process can be carried out automatically, i.e., without a force to be supplied or exerted externally by a user. The energy store advantageously stores the energy required for an automatic substance delivery in mechanical form. Such an energy store can be a spring which is installed in a tensioned state in the injection device and delivers energy by expansion. The energy is delivered to a piston rod or a pressure element, which pushes a piston into a product container. The energy store may also be provided in order to automate the process of inserting an injection needle. Alternatively, the insertion process can take place manually, i.e., exclusively by a user, without using energy stored in the injection device for this purpose.

The injection device may comprise a product container holder for accommodating a product container, wherein the product container can be held in the product container holder radially, axially, and additionally also in a rotationally fixed manner. The product container holder may be connected to the housing of the injection apparatus in an axially and rotationally fixed manner or may be movable relative to the housing during an insertion and/or needle retraction process. The product container may be a cartridge for the repeatedly detachable connection to disposable injection needles or a disposable ready-to-use syringe with an injection needle non-detachably connected thereto. The product container has a hollow-cylindrical product container portion which displaceably mounts a piston or plunger. The piston can form a sealing gap with the inner circumference of the product container portion and can be displaced in a distal direction by means of a piston rod in order to dispense product from the product container via the injection needle.

The injection device may have a needle protection sleeve which, after injection has taken place, projects distally beyond the distal end of the injection needle or is displaced relative to the housing into this position while expanding a needle protection sleeve spring, in order to prevent accidental access to the injection needle and to reduce the risk of injury. In an autoinjector, the needle protection sleeve can also serve as a trigger element for triggering the product ejection process, wherein the needle protection sleeve is displaced relative to the housing in the proximal direction for this purpose. Alternatively, the triggering of the autoinjector can be achieved by actuating a trigger button of the autoinjector, wherein the needle protection sleeve serves at least as a visual protection before the autoinjector is used.

WO 2016/205963 describes an exemplary autoinjector comprising a housing with a longitudinal axis and a product container arranged so as to be axially fixed in the housing. The autoinjector furthermore comprises a needle protection sleeve which is displaceable in a longitudinal direction between a proximal and a distal position and is coupled to a needle protection spring. A spiral spring or mainspring in which energy for the automatic discharge of product can be stored is connected via a first end to the housing and via a second end is connected in a rotationally fixed manner to a drive element in the form of a rotating threaded rod arranged coaxially with the longitudinal axis. The threaded rod engages via a thread in a propulsion member in the form of a propulsion sleeve, which propulsion member is not rotating in the housing and which propulsion sleeve moves the plunger of the product container at an approximately constant discharge rate in the distal direction during a displacement.

A mainspring as a drive is characterized by high forces and is thus suitable for a delay-free start of discharge, even with autoinjectors with a long storage period. A variable thread pitch of the threaded rod can compensate for a variable characteristic curve of the mainspring in order to ensure the most constant possible discharge force. This means that even large discharge volumes of 5 mL or more can be discharged evenly and continuously within a maximum of 60 seconds.

With an extended discharge time of more than 10 s compared to conventional autoinjectors, the user should be able to ensure that the injection is proceeding as intended at all times in order to avoid inadvertently stopping the injection prematurely.

WO 2012/173554 shows a rotating display at the proximal end of an autoinjector, which is driven by a torsion spring during discharge. The display comprises a plurality of segments with different colors for signaling an initial state prior to the commencement of discharge and a final state after discharge has taken place. The segments are visible through at least one window at the proximal end of the autoinjector, the extension of which corresponds to the angular range of a segment.

US 2014/0330214 A1 shows a display comprising a rotating disk with a plurality of sectors separated by radially extending ribs, and comprising a transparent cap with four openings in which opaque covers are placed. The latter interrupts the field of vision of the pane or ensures it only in certain directions. Before and after discharge, differently colored sectors can be recognized through the transparent portions of the cap, or sectors with different angular extensions, so that the user can deduce the condition of the device.

### SUMMARY

It is an object of the invention to create a display for an autoinjector of the type mentioned above, which can visually display to the user the persistence or progression of a discharge in a wide variety of grip positions. The display shall be configured in such a way that even in unfavorable—in particular, unintended—grip positions of a user's hand on the autoinjector, the user can recognize the persistence or progression of the discharge without having to change the grip position. The object is achieved by an autoinjector having the features of the independent claims. Embodiments of the invention are the subject matter of the dependent claims.

An autoinjector according to the invention comprises a one-piece or multi-part housing with a longitudinal axis and a pre-filled, ready-to-use syringe with a product container and an injection needle or cannula which is non-detachably fastened thereto. The ready-to-use syringe is axially non-displaceably accommodated in the housing, wherein a tip of the injection needle projects by at least one piercing depth in the distal direction beyond a distal housing end. The autoinjector furthermore comprises a torsion spring pretensioned for the single discharge of a maximum content of the product container, a drive element, a propulsion element, and a needle protection sleeve. For discharging liquid from the product container through the injection needle, the torsion spring puts the drive element into rotation about the longitudinal axis, and the rotating drive element causes a linear movement of the propulsion element in order to displace a piston in the product container. When the autoinjector is pressed against a point of injection and the injection needle is caused to pierce the point of injection as a result, the needle protection sleeve is moved in the proximal direction by an actuation stroke, thereby starting or enabling a discharge of liquid. The actuation stroke of the needle protection sleeve corresponds here to at least the piercing depth of the injection needle.

Finally, the autoinjector comprises a display for signaling the persistence or progression of a discharge, having a display element that is driven by the drive element and rotates about the longitudinal axis and has an optical contrast pattern, and a window made of a transparent and stable or load-bearing material at the proximal end of the autoinjector, through which the rotating contrast pattern is visible. The window comprises a field of vision on the contrast pattern that is completely circumferential by 360° about longitudinal axis, i.e., is not interrupted by a frame or web. The field of vision, which can be seen from all sides, avoids blind sectors or angled regions from which the view of the contrast pattern is not ensured or is interrupted. As long as even a small part of the window remains visible, i.e., even if the user clumsily covers a large part of the window with their hand holding the autoinjector, e.g., by supporting the proximal end with the heel of their hand, the rotating display element can still be recognized.

The contrast pattern is at most discretely rotationally symmetrical about the longitudinal axis, but not continuously rotationally symmetrical, so that a rotation of the contrast pattern can also be recognized. The contrast pattern comprises at least one line, a surface, or a graphic element in a color or shade of gray different from a background color or configured as a three-dimensional surface structure of the display element. The contrast pattern preferably comprises graphic elements that are repeated in the direction of rotation, such as parallel lines, which are inclined and/or curved in relation to the longitudinal axis of the autoinjector. The contrast pattern can have at least one to 60—in particular, between 8 and 16—lines or surfaces or graphic elements. The contrast pattern contains in particular one of the physiologically easily perceptible colors, green or black.

An autoinjector according to the invention is suitable for discharging the amount of product contained in the product container during a discharge time of more than 10 s—preferably more than 20 s or 30 s—which is longer than that of known autoinjectors. As a result, even with product quantities of more than 2.25 mL and preferably at least 3 mL or 4 mL, a continuous or average discharge rate is relatively low, so that the injected quantity of medicament can be continuously absorbed or resorbed by the subcutaneous tissue. Therefore, maintaining a minimum holding time between the end of the discharge and moving the autoinjector away from the puncture site is less critical. Accordingly, no visual, acoustic, or tactile signaling is required to display the end of a holding time to the user.

In a preferred embodiment, the rotating display element is driven directly or immediately—in particular, without gearing—by the drive element. The display element thus rotates at the same rotational speed as the drive element.

In a further preferred embodiment, the display element carries out less than one revolution—preferably less than half a revolution or even less than a third of a revolution—per second during discharge. This ensures that the rotational movement of the contrast pattern can be easily followed by the eye and does not blur into an indeterminate, rotationally symmetrical pattern for the viewer.

In a preferred embodiment, no non-rotating optical marker is provided either on the window or on the adjoining housing for determining a relative rotational position of the contrast pattern. The display element does not display a start or end status, nor does it provide any relative information on the progress of the discharge; it is used solely to signal the progress or persistence of the discharge.

In a preferred embodiment, the rotating display element and the housing or the window are configured to create a discharging sound, thereby additionally signaling to the user the persistence or progression of the discharge process acoustically. Preferably, the discharging sound is a continuous clicking sound, which is created by a grid on the window or on the housing and a radially or axially flexible engagement element engaging therein on the display element, or, conversely, by a rotating grid on the display element and a stationary engagement element.

In a preferred embodiment, there is no optical, acoustic, and/or tactile signaling of the end of the discharge independent of the rotation of the display element and triggered, for example, by an axial movement. Reaching the distal end position of the plunger is not displayed by a dedicated, mechanically or electronically created, signal. Only the fact that the display element is no longer rotating, together with the view of the plunger in its end position through the recesses in the housing known as viewing windows, confirms to the user that the discharging process has ended.

In a preferred embodiment, the window has a first, cylindrical region parallel to the longitudinal axis, and a second region, seamlessly adjoining the first in the proximal direction, with a constantly or continuously decreasing diameter. The first region enables a view of the display element from any lateral viewing direction perpendicular to the longitudinal axis, and the second region enables a view of the display element in the distal viewing direction from behind, or from above when the autoinjector is held vertically. The second region is rounded or constricted in a proximal direction and can end in a third region perpendicular to the longitudinal axis. The display element can have a convex display surface following the course of the window or a cone-shaped display surface with a medium inclination with respect to the longitudinal axis. Preferably, the window is rotationally symmetrical with respect to the longitudinal axis, i.e., with circular cross-sections in sectional planes perpendicular to the longitudinal axis.

In an advantageous variant, the autoinjector comprises a needle protection sleeve, which projects distally over the housing in the delivery state of the autoinjector and is pretensioned in the distal direction by a needle protection spring. When the autoinjector is pressed against a point of injection, the needle protection sleeve carries out an actuation movement in the proximal direction and, when the autoinjector is removed from the point of injection, carries out a needle protection movement in the distal direction, in order to laterally surround and radially surround the injection needle with a sleeve-shaped, preferably rotationally symmetrical, portion. The needle protection sleeve has, at one distal end, an annular flange or base as an enlarged contact surface for the tissue around the point of injection. The flange is permanently connected to the sleeve-shaped portion and is preferably formed in one piece with it. A maximum diameter of the flange is greater than a maximum diameter of the sleeve-shaped portion.

Preferably, the outer edge or the periphery of the flange is adapted to the shape of a distal opening in the housing. In the inserted state, the flange thus forms a closure for the housing. The flange is also preferably configured to be concavely curved, so that the outer edge is further distal than the transition between the flange and the sleeve-shaped portion. Compared to a flange with a strictly flat, ring-shaped contact surface with an identical outer diameter, this, firstly, makes it less likely that the attached autoinjector will tilt, and, secondly, the concentration of the pressure load on the periphery of the flange furthest away from the puncture site results in less counter-pressure in the tissue and therefore less pain for the user.

Further preferably, the autoinjector comprises a threaded rod as the drive element and a propulsion sleeve with an internal thread as the propulsion element—alternatively, a drive sleeve with an internal thread as the drive element and a threaded rod as the propulsion element—wherein the propulsion element has a groove or a cam as an axial guide element for an exclusively linear propulsion movement in the housing. The autoinjector is preferably dimensioned for accommodating a pre-filled, ready-to-use syringe comprising the product container and the injection needle, and has a filling volume of at least 3 ml.—preferably at least 5 ml.

### BRIEF DESCRIPTION OF THE DRAWINGS

Preferred embodiments of the invention are described below in connection with the appended figures. These embodiments are intended to show basic possibilities of the invention and are in no way to be interpreted as limiting. In the drawings:

FIG. 1 shows the components of an autoinjector,

FIGS. 2a and 2b show longitudinal sections through the autoinjector of FIG. 1 before injection,

FIGS. 3a and 3b show two isometric views of the display element,

FIG. 4 shows a view of the distal end of the needle protection sleeve, and

FIGS. 5a and 5b show views of a display element with a radially elastic discharge clicker.

### DETAILED DESCRIPTION

### Definitions

The term, “product,” “medicament,” or “medical substance.” in the present context includes any flowable medical formulation which is suitable for controlled administration by means of a cannula or hollow needle in subcutaneous or intramuscular tissue—for example, a liquid, a solution, a gel, or a fine suspension containing one or more medical active ingredients. A medicament can thus be a composition with a single active ingredient or a premixed or co-formulated composition with a plurality of active ingredients from a single container. The term includes in particular drugs, such as peptides (e.g., insulins, insulin-containing medicaments, GLP-1-containing preparations, as well as derived or analogous preparations), proteins and hormones, biologically obtained or active ingredients, active ingredients based upon hormones or genes, nutrient formulations, enzymes, and other substances, both in solid (suspended) or liquid form. The term also includes polysaccharides, vaccines, DNA or RNA or oligonucleotides, antibodies or parts of antibodies, as well as suitable base substances, excipients, and carrier substances.

The term, “distal,” refers to a side or direction directed towards the front, piercing-side end of the administration apparatus or towards the tip of the injection needle. In contrast, the term, “proximal,” refers to a side or direction directed towards the rear end of the administration apparatus that is opposite the piercing-side end.

In the present description, the term, “injector,” is understood to mean an apparatus with which the injection needle is removed from the tissue after a controlled amount of the medical substance has been delivered. In contrast to an infusion system, the injection needle of an injector thus does not remain in the tissue for a longer period of several hours.

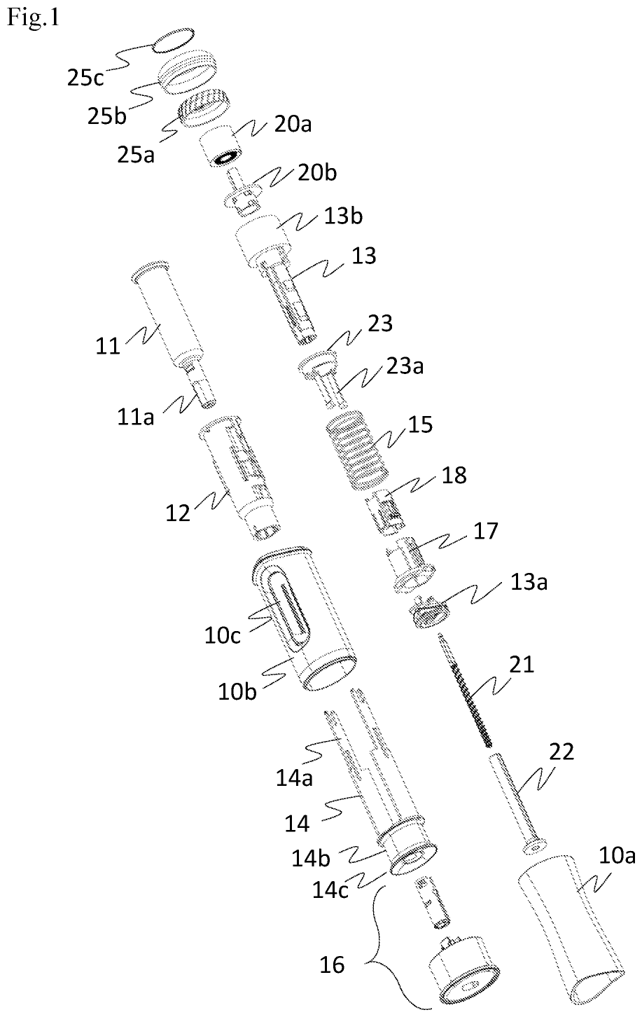

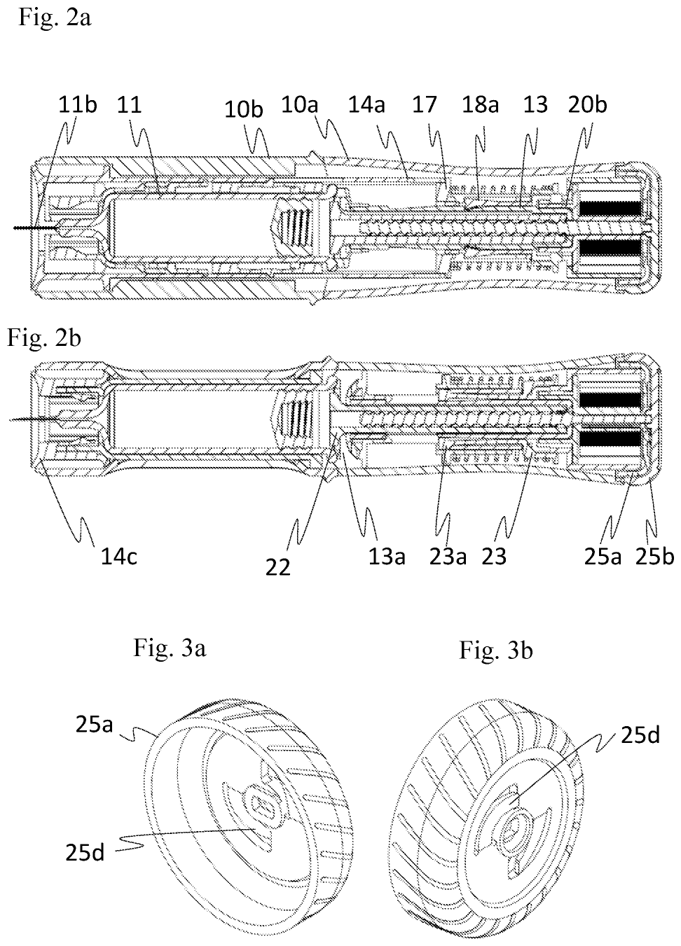

Turning to the Figures, FIG. 1 is an exploded view of the components of an autoinjector according to the invention, and FIGS. 2a and 2b illustrate two longitudinal section views of the autoinjector according to FIG. 1 rotated by 90° with respect to each other about the longitudinal axis in the inserted state and ready for discharge.

The autoinjector has a sleeve-shaped, elongated housing with a longitudinal axis L and comprising a distal housing part 10b and a proximal housing part 10a in the form of a handle that is non-detachably snap-fitted therewith. A product container in the form of a ready-to-use syringe 11 with an injection needle which is non-detachably fastened to the product container is held in a syringe holder 12, wherein the syringe holder is accommodated in the housing in an axially and rotationally fixed manner. The ready-to-use syringe 11 is pressed in a distal direction into engagement with a shoulder of the syringe holder 12 by a retaining spring portion 13a fixedly anchored in the housing part 10a. In relation to the housing part 10b, the ready-to-use syringe 11 is arranged in such a way that the tip of the injection needle projects beyond the distal end of a proximal intermediate position of the needle protection sleeve 14 by a length corresponding to the subcutaneous or intramuscular piercing depth and is at least laterally protected or covered by a needle protection sleeve 14 before and after the injection. When the injection needle is inserted into the point of injection along the longitudinal axis L, the needle protection sleeve 14 is pushed in the proximal direction by an actuation stroke and against the force of a needle protection spring 15, and thereby triggers a product discharge. For this purpose, the needle protection sleeve comprises two sleeve arms 14a, which are arranged offset or rotated by 90° about the longitudinal axis L with respect to two recesses 10c of the housing designated as viewing windows. After the injection has taken place, the needle protection sleeve 14 can be displaced relative to the housing part 10b from the actuated position along the longitudinal axis L in the distal direction to a needle protection position and can be blocked there against being pushed back again. The needle protection sleeve comprises a sleeve-shaped or hollow-cylindrical portion 14b and a flange 14c at the distal end. The cross-section of the hollow-cylindrical portion 14b is oval, as is the outer circumference of the flange, so that, in the present case, the flange has a constant width or radial extension.

A spring assembly comprises a spiral spring 20a and a spring coil 20b. The outer end of the spiral spring 20a is non-rotatably fastened to a spring sleeve 13b as part of a mechanism holder 13 fixedly anchored in the housing. The inner end of the spiral spring 20a is connected to the spring coil 20b in a rotationally fixed manner. The spring coil 20b comprises a spring shaft and a distal spring flange. The spiral spring 20a or the spring coil 20b puts a drive element 21 into a rotational movement and a propulsion element 22 into a preferably purely axial propulsion movement. For this purpose, a threaded element engages in a thread extending over the discharge stroke and having a variable thread pitch.

The ready-to-use syringe 11 comprises a cylindrical syringe body as a product container, at the distal end of which a hollow injection needle is fixedly connected to a syringe shoulder. The injection needle of the ready-to-use syringe is covered by a needle protection cap 11a, which is configured as a so-called rigid needle shield (RNS) and comprises a rubber-elastic needle protection element and a sheath made of hard plastic. The needle protection cap protects the injection needle against mechanical effects and contamination, and keeps the injection needle and the product sterile. At the distal end of the autoinjector, in the delivery state thereof, a two-part device cap or pull-off cap 16 is arranged, which is axially pulled off and/or twisted off and completely removed along with the needle protection cap 11a before the autoinjector is used.

A switching sleeve 17 is arranged in a positive-locking manner with a proximal end of the sleeve arms 14a of the needle protection sleeve 14 and with a distal end of the needle protection spring 15 and is at least partially surrounded by the latter. The switching sleeve 17 is preferably snap-fitted with the proximal end of the sleeve arms of the needle protection sleeve 14. The movement of the switching sleeve 17 in the distal direction is delimited by the retaining spring portion 13a, which in turn is snap-fitted together with the mechanism holder 13 after the switching sleeve 17 has been assembled. A locking sleeve 18 is arranged within and coaxially with the switching sleeve 17 and is coupled to the switching sleeve 17 via a saw tooth-shaped locking member 18a, which is resiliently attached to an arm pointing in the distal direction, in such a way that an actuation movement of the needle protection sleeve 14 and the switching sleeve 17 also moves the locking sleeve 18 proximally. By means of an additional proximal locking stroke of the locking sleeve 18 relative to the switching sleeve 17 into a proximal end position, the locking member 18a is reliably released by the switching sleeve 17 for movement inwards. Due to the spring effect of the arm, the locking member 18a engages behind a proximally directed edge of the autoinjector or latches into an axially fixed recess of the autoinjector and thus locks the locking sleeve 18 against a distal movement. When the autoinjector is removed from the puncture site, the switching sleeve 17 is pushed by the needle protection spring 15 in the distal direction over the locking member 18a, whereupon, as a result of the spring effect of the arm, the locking member engages behind a proximally directed edge of the switching sleeve 17 in a locking position and locks or blocks the switching sleeve and the needle protection sleeve against renewed movement in the proximal direction.

A coupling sleeve 23 with two holding cams 23a is coupled to the spring coil 20b via coupling elements. Before discharge, the holding cams 23a engage in recesses of the axially fixed mechanical holder 13 and are prevented from moving outwards by an inner circumference of the locking sleeve 18, as a result of which the coupling sleeve 23 also cannot move axially. When the discharge is triggered, the locking sleeve 18 is moved away from the position of the recesses by a proximal movement of the needle protection sleeve 14 so that the holding cams 23a can detach radially and release the coupling sleeve 23. The latter moves in the proximal direction and releases the spring coil 20b for rotation, as described in detail in patent application PCT/EP2021/076923 published as US 2023/0218827 A1. At the proximal end of the autoinjector is the display with a rotating display element 25a with a contrast pattern in the form of parallel stripes, a circumferential transparent window 25b, and a proximal, non-transparent closure 25c.

FIGS. 3a and 3b illustrate two isometric views of the display element 25a with an engagement element 25d for engagement with a non-rotating grid on the inner side of the window 25b or the closure 25d. The engagement element 25d is spring-mounted and creates a number of clicking sounds corresponding to the rasterization when the display element rotates during discharge. The display element has a shape that follows that of the window or is adapted to it, with a first, distal region and a second region adjoining it in the proximal direction. The first region is cylindrical, parallel to the longitudinal axis, while the second region has a steadily decreasing diameter.

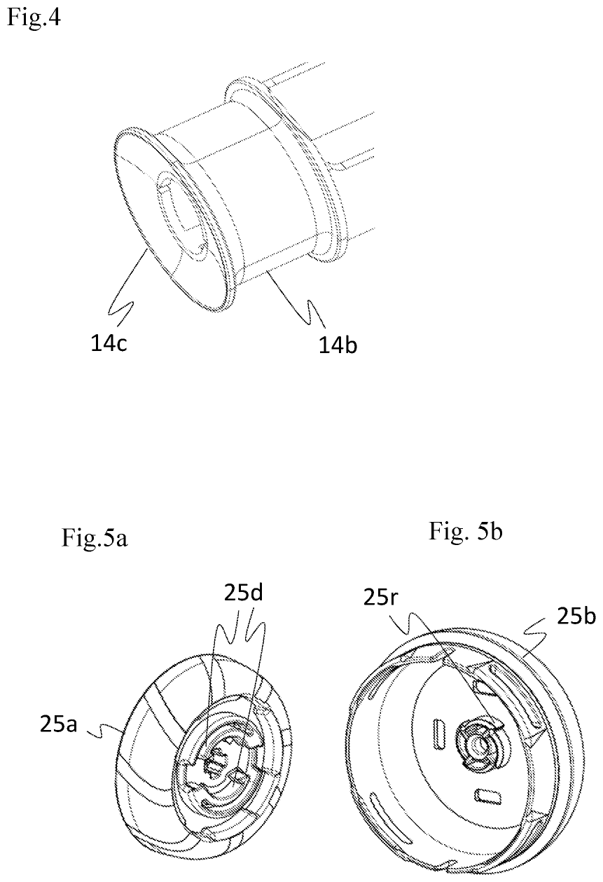

FIG. 4 shows the distal end of the needle protection sleeve with the sleeve-like portion 14b and the flange 14c. The shape of the flange is clearly visible with the concavely curved contact surface, the outer, oval edge of which projects distally from the inner edge of the contact surface at the transition to the axis-parallel portion.

FIGS. 5a and 5b illustrate isometric views of a second embodiment of the display element 25a and the window 25b. The display element has two, radially elastically or resiliently formed engagement elements 25d, which interact via latching cams at the free end with four rasterings 25r offset by 90° on the inner side of the window. When the display element rotates counter-clockwise (viewed in the distal direction), the cams are deflected outwards by the spiral-shaped guide surfaces of the counter elements, and snap radially inwards again at the end of the guide surfaces. This creates a clicking noise and at the same time prevents clockwise rotation of the display element due to the shape of the engagement. In contrast to the axial springing of the engagement element in FIGS. 3a and 3b, the spring pre-tensioning and the click behavior are independent of the exact axial position of the display element, so a certain tolerance or play in this respect is not critical. The small friction radius of the radially deflecting engagement elements also leads to low friction losses, and thus to a minimal influence on the discharge behavior.

The contrast pattern described here can be printed on the base body of the display element using the pad printing process or thermal transfer printing. The contrast pattern can also be produced in different colors using 2-component injection molding. The two components are preferably split, wherein the first component—preferably made of PBT—contains the functional elements, such as the snaps. This means that the functional elements are always created with the same material and preferably in the same color. The visual, aesthetic elements such as the contrast pattern can be created with the second component—preferably made of colored ABS or PP—and vary in color.

The contrast pattern can also be created using a laser process—preferably a UV laser—by locally removing material from the display element on the visible surface, thereby creating a modified structure—particularly a gray one. The surface of the display element can also be melted using a laser process and a suitable choice of material, as a result of which pigments become visible, or the plastic foams up due to its ingredients, thereby modifying the optical properties in a contrasting manner. Alternatively, material can be removed locally and finely structured by laser or erosion on the previously polished visible surface of the injection mold, as a result of which structures—in particular, roof or cube structures—can be created, which are later transferred to the plastic part during injection molding. Such surface structures can have a depth or raised surface of 0.01 to 0.5 mm. This is an advantageous configuration for reasons of sustainability, since, for the component, no different materials are mixed, no paint is applied to the material, and the material is not modified locally by laser marking.

The contrast pattern can also be created by means of openings in the visible surface of the display element. The openings allow a view into the dark-looking interior of the autoinjector—in particular, onto the metallic spiral torsion spring.

### LIST OF REFERENCE SIGNS

10a, 10b Housing part
10c Recess
11 Ready-to-use syringe
11a Needle protection cap
11b Injection needle
12 Syringe holder
13 Mechanical holder
13a Retaining spring portion
13b Spring sleeve
14 Needle protection sleeve
14a Arm
16 Device cap
17 Switching sleeve
18 Locking sleeve
18a Locking member
20a Spiral spring
20b Spring coil
21 Drive element
22 Propulsion element or member
23 Coupling sleeve
23a Holding cam
25a Display element
14b Portion
14c Flange
15 Needle protection spring
25b Window
25c Closure
25d Engagement element

What is claimed is:

1. An autoinjector, comprising:
a housing having a longitudinal axis configured to accommodate a product container;
a propulsion member;
a drive element configured to be rotatable to move the propulsion member along the longitudinal axis for automatically discharging a liquid product contained in the product container through an injection needle;
a display configured to signal a progression of a discharge, the display comprising a rotating display element configured to be driven by the drive element, a rotatable optical contrast pattern, and a window at a proximal end of the autoinjector through which the rotatable contrast pattern is visible,
wherein the window is configured as an uninterrupted window circumferentially surrounding the longitudinal axis.

2. The autoinjector of claim 1, wherein the rotating display element is configured to be driven directly by the drive element.

3. The autoinjector of claim 2, wherein the display element carries out less than one revolution per second during the discharge.

4. The autoinjector of claim 1, wherein no optical marking is provided for determining a rotational position of the contrast pattern.

5. The autoinjector of claim 1, wherein the rotating display element is configured to generate a discharging sound during the discharge.

6. The autoinjector of claim 5, wherein the discharging sounds comprised clicking sounds generated by an axially resilient engagement element.

7. The autoinjector of claim 6, wherein no independent optical, acoustic, and/or tactile signaling of an end of the discharge is provided.

8. The autoinjector of claim 1, wherein the window comprises a first cylindrical region parallel to the longitudinal axis and a second region seamlessly adjoining the first cylindrical region with a constantly decreasing diameter in a proximal direction.

9. The autoinjector of claim 1, further comprising:
a needle protection sleeve projecting distally over the housing in a delivery state of the autoinjector,
wherein when the autoinjector is pressed against a point of injection, the needle protection sleeve carries out an actuation movement in a proximal direction and, when the autoinjector is removed from the point of injection, carries out a needle protection movement in a distal direction in order to laterally surround the injection needle with a sleeve-shaped portion, and
wherein the needle protection sleeve comprises an annular flange at a distal end for contact with the point of injection with a maximum diameter that is greater than a maximum diameter of the sleeve-shaped portion.

10. The autoinjector of claim 9, wherein an outer edge of the flange is adapted to a distal opening in the housing.

11. The autoinjector of claim 9, wherein the flange is configured to be concavely curved.

12. The autoinjector of claim 9, wherein the drive element is a threaded rod and the propulsion member comprises a propulsion sleeve and an axial guide element for an exclusively linear propulsion movement in the housing.

13. The autoinjector of claim 9, wherein the product container and the injection needle is configured as a pre-filled, ready-to-use syringe with a filling volume of at least 3 ml.
